(19) United States
(12) Patent Application Publication
(10) Pub. No.: US 2004/0097528 A1
(43) Pub. Date: May 20, 2004

(54) Crystalline solid famciclovir forms I, II, III and preparation thereof

(76) Inventors: Ben-Zion Dolitzky, Petach Tiqva (IL); Shlomit Wizel, Petah Tiqva (IL); Ofer Reany, Lower Gallilee (IL); Jenny Shammai, Kiryat Yam (IL)

Correspondence Address: KENYON & KENYON, ONE BROADWAY, NEW YORK, NY, 10004, US

(21) Appl. No.: 10/649,399

(22) Filed: Aug. 26, 2003

Related U.S. Application Data
(60) Provisional application No. 60/406,173, filed on Aug. 26, 2002.
(60) Provisional application No. 60/422,243, filed on Oct. 29, 2002.

(51) Int. Cl. C07D 473/14; A61K 31/52

(52) U.S. Cl. 514/263.4; 544/277

(57) ABSTRACT

The present invention provides novel crystalline solid anhydrous forms of famciclovir, denominated form I, II and III, as well as their preparations thereof.

### CROSS-REFERENCE TO RELATED APPLICATION

This application claims the benefit under 35 U.S.C. § 1.119(e) of Provisional Application Serial Nos. 60/406,173 filed Aug. 26, 2002 and 60/422,243 filed Oct. 29, 2002, the disclosure of which is incorporated by reference in its entirety herein.

### FIELD OF THE INVENTION

The present invention relates to crystalline solid forms of famciclovir and processes for their preparation.

### BACKGROUND OF THE INVENTION

PCT WO 97/29108 and EP 0 885 223 B1 describe an anhydrous form and a monohydrate form of famciclovir. Crystallographic data for famciclovir monohydrate are given in Nucleosides & Nucleotides 9(4): 499-513 (1990).

Different crystalline solid forms of famciclovir may have different solid state physical properties, thermal stability, cost of preparation, dissolution characteristics and bioavailability.

The discovery of a new crystalline solid form of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. It is clearly advantageous when this repertoire is enlarged by the discovery of new crystalline solid forms of a useful compound. For a general review of polymorphs and the pharmaceutical applications of polymorphs; see G. M. Wall, Pharm Manuf. 3, 33 (1986); J. K. Haleblian and W. McCrone, J. Pharm. Sci., 58, 911 (1969); and J. K. Haleblian, J. Pharm. Sci., 64, 1269 (1975), all of which are incorporated herein by reference.

Solid state physical properties of a polymorph compound include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a polymorph compound is its rate of dissolution in aqueous fluid. The rate of dissolution of an active ingredient in a patient's stomach fluid can have therapeutic consequences since it may impose an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and storage stability. These practical physical characteristics are influenced by the conformation and orientation of molecules in the unit cell, which defines a particular polymorphic form.

The polymorphic form may also give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

There is a constant need to search for better crystalline solid forms of famciclovir that can provide a better pharmaceutical composition, e.g., a form that is suitable for use in tablet or capsule due to good stability, handling qualities and like properties.

### SUMMARY OF THE INVENTION

The present invention provides three novel crystalline forms of famciclovir.

The present invention provides a crystalline form of famciclovir, denominated form I, characterized by XRD peaks at 15.5 and 15.9±0.2 deg. 2θ.

The present invention provides a crystalline form of famciclovir, denominated form II, characterized by XRD peaks at 16.2 and 16.4±0.2 deg. 2θ.

The present invention provides a crystalline solid form of famciclovir, denominated form III, that is a methanol solvate, characterized by XRD peaks at 6.6 and 13.0±0.2 deg. 2θ.

The present invention also provides a crystalline form of famciclovir, form I, characterized by XRD peaks at 15.5 and 15.9±0.2 deg. 2θ, wherein the crystalline solid form contains less than about 5% wt of other famciclovir forms.

The present invention provides a process for preparing a crystalline famciclovir form I, comprising the steps of: a) triturating anhydrous famciclovir in an organic solvent selected from the group consisting of isopropyl alcohol, acetonitrile, and diethylether; and b) isolating famciclovir form I.

The present invention provides a process for preparing crystalline famciclovir form I that contains less than about 5% wt of other famciclovir crystalline forms, comprising the steps of: a) triturating anhydrous famciclovir in an organic solvent selected from the group consisting of isopropyl alcohol, acetonitrile, and diethylether; and b) isolating the famciclovir form I, wherein the isolated crystalline solid famciclovir form I.

The present invention provides a process for preparing crystalline famciclovir form I, comprising the steps of: a) heating a crystalline solid famciclovir form III to about 40° C. to about 90° C.; especially about 60° to about 70° C., and b) isolating crystalline solid famciclovir form I.

The present invention provides a process for preparing crystalline solid famciclovir form I, comprising the steps of: a) heating famciclovir monohydrate to about 40° C. to about 80° C.; especially about 60° to about 70° C., and b) isolating crystalline solid famciclovir form I.

The present invention provides a process for preparing crystalline solid famciclovir form I, comprising the steps of: a) heating crystalline solid famciclovir form II or a mixture of crystalline solid famciclovir I and crystalline solid famciclovir form II to about 40° C. to about 70° C.; and b) isolating famciclovir form I.

The present invention provides a process for preparing a crystalline solid famciclovir form I, which contains less than about 5% wt of other famciclovir crystalline forms, comprising the steps of: a) heating crystalline solid famciclovir selected from the group comprising of form III, monohydrate, form II and a mixture of form I and form II to about 40° C. to about 90° C.; and b) isolating famciclovir form I.

The present invention provides a process for preparing crystalline solid famciclovir form I, comprising the steps of: a) providing a solution of famciclovir form II in an organic solvent selected from the group consisting of dichloromethane, chloroform, acetonitrile, ethylacetate, acetone, THF, diethyl ether/dichloromethane mixture, dichloromethane/toluene mixture, ethylacetate/toluene mixture, acetonitrile/toluene mixture, dimethylacetamide and isopropylalcohol; b) cooling the solution; and c) isolating crystalline solid famciclovir form I.

The present invention provides a process for preparing crystalline solid famciclovir form II, comprising the steps of: a) providing a solution of famciclovir in an organic solvent selected from the group consisting of ethanol and n-butanol; b) cooling the solution; and c) isolating crystalline solid famciclovir form II.

The present invention provides a process for preparing mixture of crystalline solid famciclovir form II and crystalline solid famciclovir form I, comprising the steps of: a) providing a solution of famciclovir in an organic solvent selected from the group consisting of chloroform, ethylacetate, diethyl ether/dichloromethane mixture, tetrahydrofuran, acetonitrile/toluene mixture, dimethylacetamide, and isopropanol; b) cooling the solution; and c) isolating the mixture of crystalline solid famciclovir form II and crystalline solid famciclovir form I.

The present invention provides a process for preparing crystalline solid famciclovir form III, comprising the steps of: a) triturating anhydrous famciclovir in methanol; and b) isolating crystalline solid famciclovir form III.

The present invention provides a process for preparing a mixture of famciclovir form III and crystalline solid famciclovir form I, comprising the steps of: a) triturating anhydrous famciclovir in ethanol; and b) isolating the mixture of crystalline solid famciclovir form III and crystalline solid famciclovir form I.

The present invention provides a process of preparing a crystalline solid famciclovir monohydrate, comprising the steps of: a) providing a solution of famciclovir in an organic solvent selected from the group consisting of acetonitrile, ethyl acetate, acetone, isopropyl alcohol, tetrahydrofuran, ethanol/water mixture, acetone/water mixture, DMF/water mixture, DMA/water mixture, acetonitrile/water mixture, methanol/water mixture, tetrahydrofuran/water mixture, and isopropyl alcohol/water mixture; b) cooling the solution; and c) isolating the crystalline solid famciclovir monohydrate.

The present invention provides a process for preparing a mixture of crystalline solid famciclovir form III and crystalline solid famciclovir monohydrate, comprising the steps of: a) triturating anhydrous famciclovir in an organic solvent selected from the group consisting of isopropyl alcohol and ethanol; and b) isolating the mixture of crystalline solid famciclovir form I and crystalline solid famciclovir monohydrate.

The present invention provides a process for preparing a crystalline solid famciclovir form I by drying a mixture of famciclovir form I and crystalline solid famciclovir monohydrate.

The present invention also provides pharmaceutical compositions containing famciclovir crystalline forms I, II, III or mixtures thereof.

The present invention also provides a method of treating a human in need of treatment with famciclovir comprising administering to the human an effective amount of a pharmaceutical composition containing one or more of the novel crystalline solid forms of famciclovir of the present invention.

### BRIEF DESCRIPTION OF THE FIGURES

FIG. 1 depicts a XRD diffractogram of crystalline solid famciclovir form I.

FIG. 2 depicts a XRD diffractogram of crystalline solid famciclovir form II.

FIG. 3 depicts a XRD diffractogram of crystalline solid famciclovir form III (a methanol solvate).

FIG. 4 depicts a DSC thermogram of crystalline solid famciclovir form II.

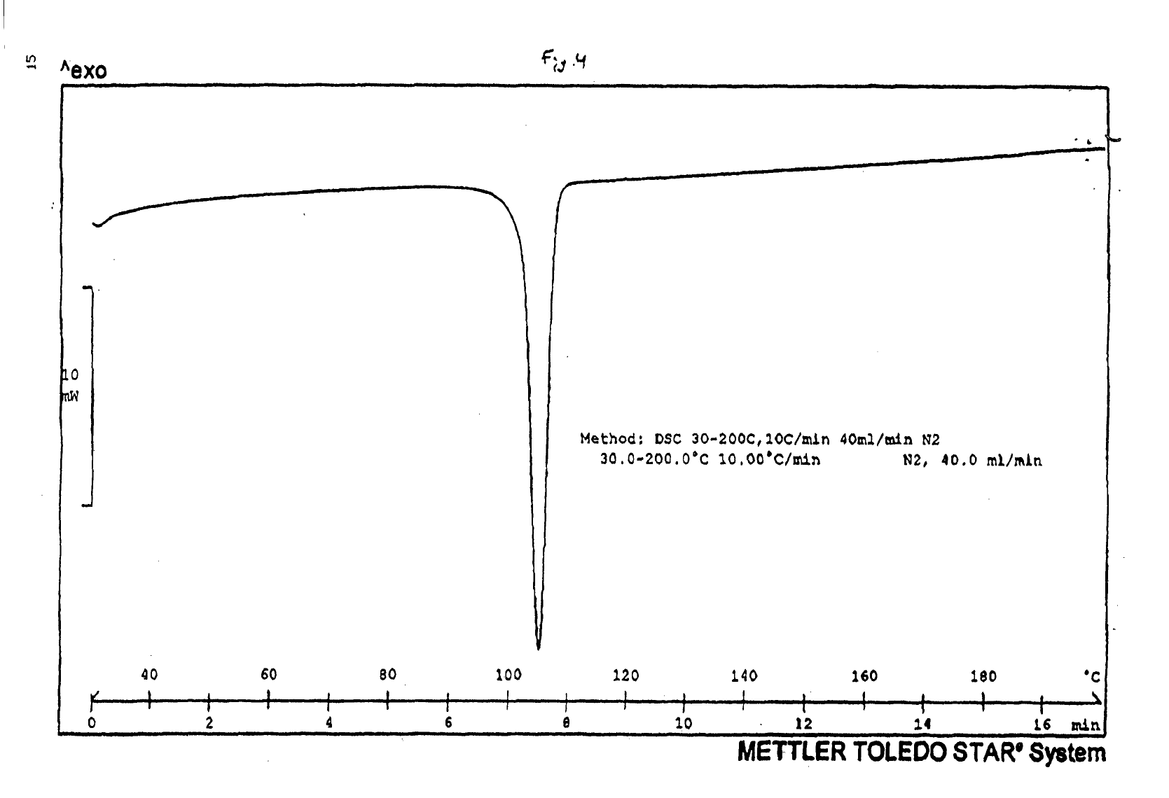

FIG. 5 depicts a DSC thermogram of crystalline solid famciclovir form III (a methanol solvate).

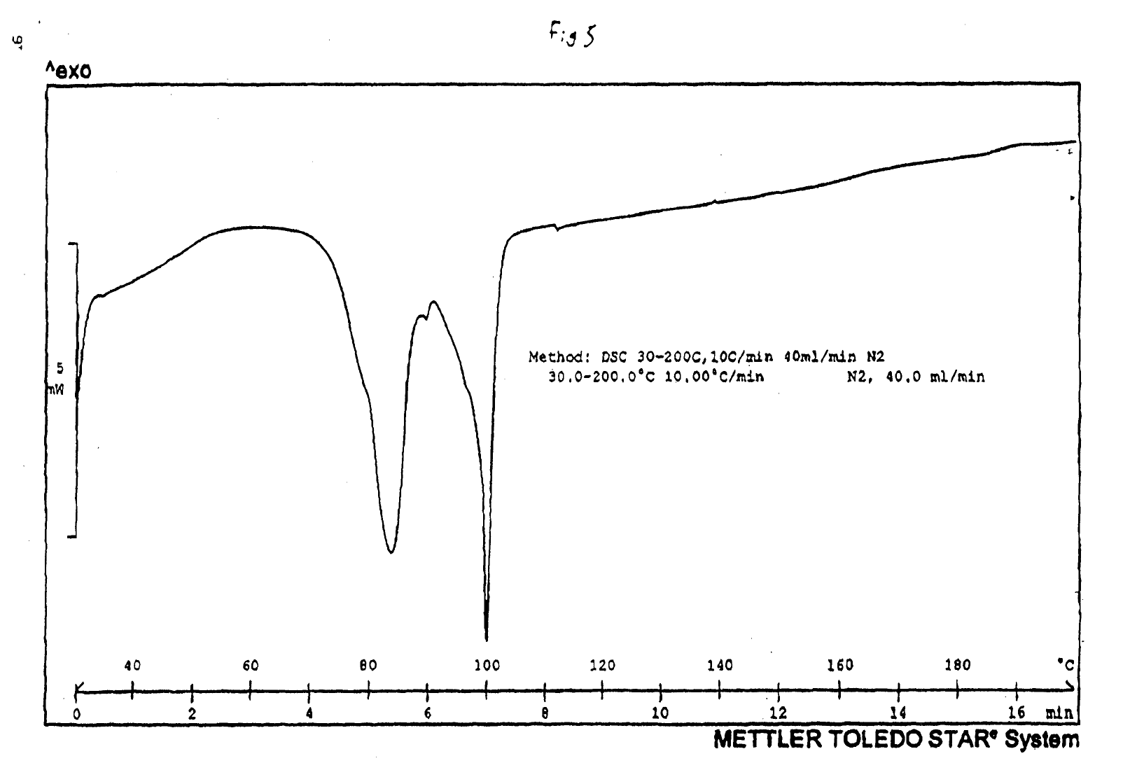

FIG. 6 depicts a TGA thermogram of crystalline solid famciclovir form III (a methanol solvate).

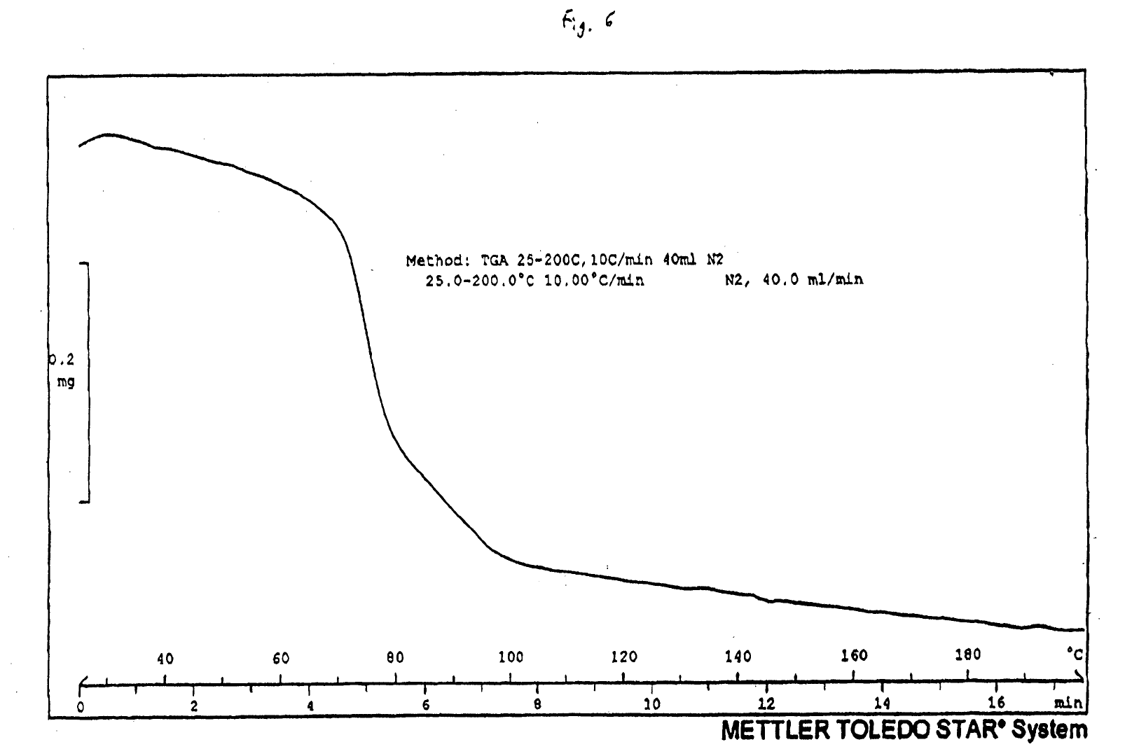

### DETAILED DESCRIPTION OF THE INVENTION

The present invention provides two novel crystalline solid anhydrous forms of famciclovir, denominated form I and form II. The present invention also provides one solvated crystalline solid form of famciclovir, denominated form III. The physical characterization of these famciclovir forms was performed using X-ray powder diffraction. The new crystalline forms may also be characterized by thermogravimetric analysis.

All powder X-ray diffraction patterns were obtained by methods known in the art using a Scintag X'TRA X-ray powder diffractometer, equipped with a solid state Si(Li) detector thermoelectrically cooled, at scanning speed of 3° min.−1 and a scanning range of 2-40 degrees two-theta. Copper radiation of λ=1.5418 was used.

Differential scanning calorimetry (DSC) was performed with a model 827e, Mettler Toledo, sample weight: 3-5 mg, heating rate: 10° C./min, 3 holes in the crucibles.

TGA, thermogravimetry analysis, was performed with a Mettler TG 50, heating rate 10° C./min, sample weight 7-15 mg. TGA is a measure of the thermally induced weight loss of a material as a function of the applied temperature. TGA is restricted to transitions that involve either a gain or a loss of mass, and has been used to study desolvation processes and compound decomposition.

The term “anhydrous famciclovir” will be understood to include a mixture of solid crystalline famciclovir form I and solid crystalline famciclovir form II, both anhydrous.

The term “substantially pure famciclovir” refers to a crystalline solid famciclovir form that is substantially pure and free of other forms of famciclovir. Purity was assessed according to the X-ray peaks. For example, a substantially pure crystalline solid famciclovir form I refers to a famciclovir polymorph that is free (less than 5% (w/w)) of other crystalline solid forms of famciclovir including famciclovir form II. Likewise, a substantially pure crystalline solid famciclovir form II refers to a famciclovir polymorph that is free (less than 5% (w/w)) of other crystalline solid forms of famciclovir including famciclovir form II. Preferably, a substantially pure crystalline solid famciclovir contains less than 1% (w/w) of other crystalline forms. The expression “% wt: is used herein to refer to % on a weight basis (wt/wt).

Crystalline solid famciclovir form III is a solvate that can be a methanol solvate or an ethanol solvate. Both of these solvates have the same physiochemical properties and the same XRD pattern.

Trituration refers to the process of mixing a solvent with a solid powder.

### Physical Characterization

### XRD Studies

Crystalline Solid Famciclovir form I: The obtained crystalline solid famciclovir form I was characterized by XRD peaks at 15.5 and 15.9±0.2 deg. 2θ. Other XRD peaks that further characterize the form are observed at 8.2, 10.4, 14.5, 17.0, 17.7, 19.5, 20.6, 21.1, 22.3, 23.0, 23.9, 24.4, 25.6, 26.5, 28.6, 29.0 and 32.6±0.2 deg. 2θ.

Crystalline Solid Famciclovir form II: The obtained crystalline solid famciclovir form II was characterized by XRD peaks at 16.2 and 16.4±0.2 deg. 2θ. Other XRD peaks that further characterize the form are observed at 8.3, 14.6, 17.0, 17.8, 19.3, 19.7, 20.7, 21.2, 24.5, 25.6, 26.5, 28.5 and 32.6±0.2 deg. 2θ.

Crystalline Solid Famciclovir form III: The obtained crystalline solid famciclovir form III (a methanol solvate) was characterized by the XRD peaks at 6.6 and 13.0±0.2 deg. 2θ. Other XRD peaks that further characterize the form are observed at 15.9, 16.7, 17.9, 18.4, 19.1, 19.6, 22.1, 22.8, 23.1, 24.5, 25.0, 26.2, 28.4 and 28.8±0.2 deg. 2θ.

### Thermogravimetric Analysis

Crystalline Solid Famciclovir form I and form II: The obtained crystalline solid famciclovir form I or form II cannot be distinguished by their DSC profile. Both crystalline solid famciclovir forms showed one endothermic peak at about 104° C.

Crystalline Solid Famciclovir form III: The obtained crystalline solid famciclovir form III (a methanol solvate) was characterized by an endothermic peak at 84° C. followed by another endothermic peak at 100° C.

### TGA Analysis

Famciclovir form III: TGA analysis of the obtained crystalline solid famciclovir form III (a methanol solvate) showed a weight loss step of about 3.6% due to the release of solvent from the crystals.

### Preparation Procedures for Famciclovir Polymorphic Forms Crystalline Solid Famciclovir Form I

Crystalline solid famciclovir form I was prepared by trituration of the anhydrous form with isopropyl alcohol, acetonitrile or diethylether. Preferably, the crystalline solid famciclovir form I, obtained contains less than about 5% wt of other famciclovir crystalline forms, more preferably less than about 1% wt of other famciclovir crystalline forms.

Since crystalline solid famciclovir form I was often crystallized with other crystal forms, usually crystalline solid famciclovir form II, the trituration with an organic solvent such as isopropylalcohol, acetonitrile or diethylether can be used to convert a mixture of crystalline solid famciclovir form I and crystalline solid famciclovir form II into a substantially pure crystalline solid famciclovir form I.

Crystalline solid famciclovir form I can be prepared, usually in a mixture with other crystal forms, by crystallization from various organic solvents such as dichloromethane, chloroform, acetonitrile, ethylacetate, acetone, THF, diethyl ether/dichloromethane mixture, dichloromethane/toluene mixture, ethylacetate/toluene mixture, acetonitrile/tolene mixture, dimethylacetamide and isopropylalcohol.

Crystalline solid famciclovir form I was also prepared by heating a monohydrate form or a mixture of monohydrate and form I to about 40° C. to about 90° C., preferably to about 60° C. to about 70° C.

Crystalline solid famciclovir form I can be prepared by heating famciclovir form II or a mixture of crystalline solid famciclovir form I and crystalline solid famciclovir form II to about 40° C. to about 70° C.

Crystalline solid famciclovir form I can be prepared by heating crystalline solid famciclovir form III to about 40° C. to about 90° C., preferably to about 60° C. to about 70° C.

Crystalline solid famciclovir form I, which contains less than about 5% wt of other famciclovir crystalline forms, preferably less than about 1% wt of other famciclovir crystalline forms, can also be prepared by heating crystalline solid famciclovir selected from the group comprising of form III, monohydrate, form II and a mixture of form I and form II to about 40° C. to about 90° C.

The function and advantage of these and other embodiments of the present invention will be more fully understood from the examples below. The following examples are intended to illustrate the benefits of the present invention, but do not necessarily exemplify the full scope of the invention.

### EXAMPLES

### General

### Example 1

### Preparation of Crystalline Solid Famciclovir Form I by Crystallization

Method A: Famciclovir (a mixture of crystalline solid famciclovir form I and form II) (3 grams) was dissolved in a minimum volume of solvent (as depicted in Table 1) while stirring. If necessary, the mixture was warmed for a short time until no precipitate was observed. The solution was then cooled to room temperature and allowed to stand overnight. If required, the solution was left to stand for a longer period of time. The crystals (a substantially pure crystalline solid famciclovir form I) were filtered off and dried.

Method B: Famciclovir (a mixture of crystalline solid form I and form II) (3 grams) was dissolved in a minimum volume of solvent (as depicted in Table 1). The mixture was heated while adding the solvent (in 1 ml portions) until the solution was clear and no precipitate was observed. The solution was then cooled to room temperature and left to stand overnight. If required, the solution was left to stand for a longer period of time. The crystals (a substantially pure crystalline solid famciclovir form I) were filtered off and dried.

Method C: Famciclovir (a mixture of crystalline solid form I and form II) (3 grams) was dissolved in 30-40 ml of solvent (as depicted in Table 1) at room temperature. An anti-solvent was added to the mixture. The resulting solution was allowed to stand overnight. If required, the resulting solution was left to stand for a longer period of time. The crystals (a substantially pure crystalline solid famciclovir form I) were filtered off and dried.

TABLE 1

| Exp. | | | Methods | Resulting |
|---|---|---|---|---|
| No. | Solvent System | Drying Conditions | used | Form |
| 1 | DCM-7 ml | 40° C. under vacuum | A | I |
| 2 | CHCl3-6 ml | 40° C. under vacuum | A | I + II |
| 3 | ACN-20 ml | 40° C. under vacuum | A | I + II |
| 4 | EA-20 ml | 40° C. under vacuum | A | I + II |
| 5 | Acetone-20 ml | 40° C. under vacuum | A | I + II |
| 6 | DEE-50 ml | 40° C. under vacuum | C | I + II |
| | DCM-24 ml | | | |
| 7 | THF-20 ml | 40° C. under vacuum | B | I + II |
| 8 | CAN-30 ml | 40° C. under vacuum | C | I + II |
| | Toluene-30 ml | | | |
| 9 | IPA-25 ml | 40° C. under vacuum | B | I + II |
| 10 | IPA-6 ml | 40° C. under vacuum | B | I + II |
| 11 | IPA-20 ml | 40° C. under vacuum | B | I + II |
| 12 | IPA-9 ml | 40° C. under vacuum | B | I + II |
| 13 | DCM-40 ml | 40° C. under vacuum | C | I + II |
| | Toluene-21 ml | | | |
| 14 | EA-13 ml | 40° C. under vacuum | C | I + II |
| | Toluene-7 ml | | | |
| 15 | DMA-3 ml | 40° C. under vacuum | B | I + II |

DCM is dichloromethane;
ACN is acetonitrile;
EA is ethyl acetate;
DEE is diethylether;
DMA is dimethyl acetamide.

### Example 2

### Substantially Pure Crystalline Solid Famciclovir Form I

A mixture of crystalline solid famciclovir form I and form II (3.02 grams) was triturated in isopropyl alcohol (20 drops) in a sealed Erlenmeyer flask at room temperature under vigorous stirring. After five days, the triturated material (3.15 grams) was collected. A substantially pure famciclovir form I was obtained as wet sample and after drying at 65° C. under vacuum for two hours.

### Example 3

### Famciclovir Form I

Crystalline solid famciclovir form II was heated to 40° C. under vacuum for 2 hours. A substantially pure crystalline solid famciclovir form I was obtained.

### Crystalline Solid Famciclovir Form II

Crystalline solid famciclovir form II was crystallized from ethanol. Crystalline solid famciclovir form II was also crystallized from n-butanol.

Crystalline solid famciclovir form II was crystallized as a mixture with other crystal form (usually form I) from chloroform. The crystallization was also performed with ethylacetate, diethyl ether/dichloromethane, tetrahydrofuran, acetonitrile/toluene, dimethyl acetamide and isopropanol.

### Example 4

### Crystalline Solid Famciclovir Form II

Famciclovir (3 grams) (a mixture of crystalline solid famciclovir form I and II) was dissolved in EtOH (20 mL) at 66° C. The clear solution was left standing overnight. Filtration gave 2.4 grams of wet famciclovir form II crystals.

Drying the crystalline solid famciclovir form II at 65° C. in vacuum for 2 hours resulted in a mixture of crystalline solid famciclovir form I and form II.

### Example 5

### Crystalline Solid Famciclovir Form II

Famciclovir (3 grams) (a mixture of crystalline solid form I and II) was dissolved in n-BuOH (20 mL) at 63° C. Upon cooling of the clear solution, crystallization took place within an hour. The slurry was left to stir for 72 hours. Filtration gave 3.2 grams of wet famciclovir crystalline solid form II crystals as determined by XRD.

### Example 6

### Crystalline Solid Famciclovir Form II

Famciclovir (3 grams) (a mixture of crystalline solid form I and II) was dissolved in n-BuOH (30 mL) at 63° C. The clear solution was left at ambient temperature overnight. Filtration gave 3.1 grams wet famciclovir form II crystals as determined by XRD. The drying of wet famciclovir form II was performed at 40° C. under vacuum for two hours to obtain substantially pure crystalline solid famciclovir form II crystals.

### Crystalline Solid Famciclovir form III

Crystalline solid famciclovir form III was obtained by trituration of anhydrous famciclovir with MeOH. A mixture of crystalline solid famciclovir form III and form I was obtained by trituration of anhydrous famciclovir with ethanol.

A mixture of crystalline solid famciclovir form III and famciclovir monohydrate was obtained by trituration of anhydrous famciclovir in ethanol.

### Example 7

### Crystalline Solid Famciclovir Form III

Famciclovir (3 grams) (a mixture of crystalline solid form I and II) was triturated at room temperature in MeOH (20 drops) in a sealed Erlenmeyer flask under vigorous stirring. After five days, the triturated material was filtered. 3.5 grams of wet famciclovir form III crystals were obtained.

Drying was performed at 65° C. under vacuum for two hours to obtain famciclovir crystalline solid form I.

### Example 8

### Crystalline Solid Famciclovir Form III

Famciclovir (3 grams) (a mixture of crystalline solid famciclovir form I and II) was triturated at room temperature in EtOH (20 drops) in a sealed Erlenmeyer flask under vigorous stirring. After five days, the triturated material was filtered. 3.2 grams of wet material were obtained. XRD analysis showed the presence of crystalline solid famciclovir form III and famciclovir form I. Drying was performed at 65° C. under vacuum for two hours to obtain substantially pure crystalline solid famciclovir form I crystals.

### Example 9

### Crystalline Solid Famciclovir Form III

A suspension of famciclovir (3 grams) (a mixture of crystalline solid famciclovir form I and II) in EtOH (30 mL) was stirred at room temperature overnight. Filtration gave 2.2 grams wet solid. XRD analysis showed the presence of famciclovir crystalline solid form III and monohydrate. Drying was performed at 65° C. under vacuum for two hours to obtain substantially pure famciclovir crystalline solid form I crystals.

### Example 10

### Famciclovir Monohydrate

3 grams of famciclovir (a mixture of crystalline solid famciclovir form I and II) were dissolved in a minimum volume of solvent mixture (water:DMF=1:1). The solvent mixture was aliquoted into 1 ml portion and the 1 ml solvent mixture was heated until a clear solution was obtained. The solution was then cooled to room temperature and left to stand overnight to induce formation of crystals. The crystals formed were filtered. The crystals formed are famciclovir monohydrate.

### Example 11

### Famciclovir Monohydrate

3 grams of famciclovir were suspended in 30 ml isopropyl alcohol. The suspension was stirred vigorously at room temperature overnight. The crystals formed were filtered. The crystals formed are a mixture of famciclovir monohydrate and crystalline solid famciclovir form I.

### Pharmaceutical Formulations and Dosages

Famciclovir may be formulated into a variety of pharmaceutical compositions and dosage forms that are of therapeutic use in treating patients.

Pharmaceutical compositions of the present invention contain at least one crystalline solid famciclovir form. In addition to the active ingredient(s), famciclovir pharmaceutical compositions of the present invention may contain one or more pharmaceutically-acceptable excipients. Excipients are added to the composition for a variety of purposes.

Diluents increase the bulk of a solid pharmaceutical composition and may make a pharmaceutical dosage form containing the composition easier for the patient and caregiver to handle. Diluents for solid compositions include, for example, microcrystalline cellulose (e.g. AVICEL®, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Solid pharmaceutical compositions that are compacted into a dosage form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®) and starch.

Glidants can be added to improve the flow properties of non-compacted solid compositions and improve the accuracy of dosing. Excipients that may function as glidants include colloidal silicon dixoide, magnesium trisilicate, powdered cellulose, starch, talc and tribasic calcium phosphate.

When a dosage form such as a tablet is made by compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The solid composition of the present invention can be administered to a human in need of treatment. The dosages may be conveniently presented in unit dosage form and prepared by any of the methods well-known in the pharmaceutical arts.

Dosage forms include solid dosage forms like tablets, powders, capsules, suppositories, sachets, troches and lozenges as well as liquid syrups, suspensions and elixirs. An especially preferred dosage form of the present invention is a tablet.

Tablets, capsules, lozenges and other unit dosage forms preferably contain famciclovir in a dosage level of from about 50 to about 300 mg, more preferably from about 100 mg to about 200 mg.

What is claimed is:

1. A crystalline solid famciclovir form I, characterized by a XRD pattern with peaks at 15.5 and 15.9±0.2 deg. 2θ.

2. The crystalline solid famciclovir of claim 1, further characterized by a XRD pattern with peaks at 8.2, 10.4, 14.5, 17.0, 17.7, 19.5, 20.6, 21.1, 22.3, 23.0, 23.9, 24.4, 25.6, 26.5, 28.6, 29.0 and 32.6±0.2 deg. 2θ.

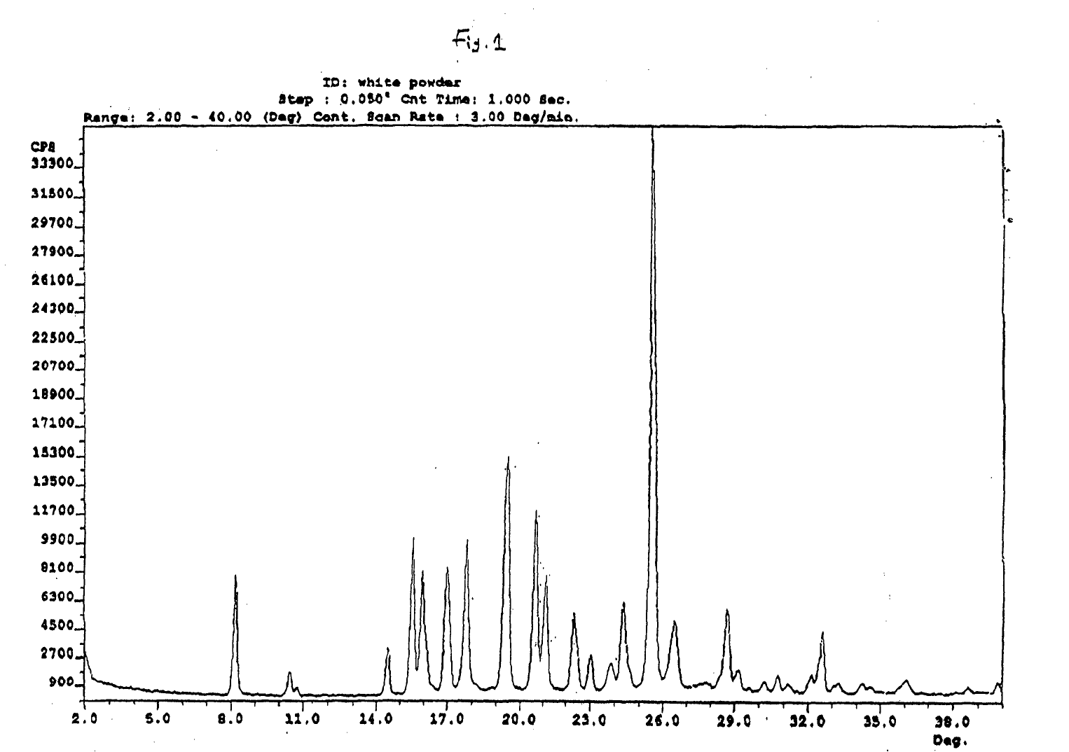

3. The crystalline solid famciclovir of claim 2, further characterized by a XRD pattern as substantially depicted in FIG. 1.

4. The crystalline solid famciclovir of any of claims 1-3, wherein the crystalline solid famciclovir contains less than about 5% wt of other famciclovir crystalline forms.

5. The crystalline solid famciclovir of any of claims 1-3, wherein the crystalline solid famciclovir contains less than about 5% wt of form II.

6. The crystalline solid famciclovir of claim 4, wherein the crystalline solid famciclovir contains less than about 1% wt of other famciclovir crystalline forms.

7. The crystalline solid famciclovir of claim 6, wherein the crystalline solid famciclovir contains less than about 1% wt of form II.

8. A crystalline solid famciclovir form II, characterized by a XRD pattern with peaks at 16.2 and 16.4±0.2 deg. 2θ.

9. The crystalline solid famciclovir of claim 8, further characterized by a XRD pattern with peaks at 8.3, 14.6, 17.8, 19.7, 20.7, 21.2, 24.5 and 25.6±0.2 deg. 2θ.

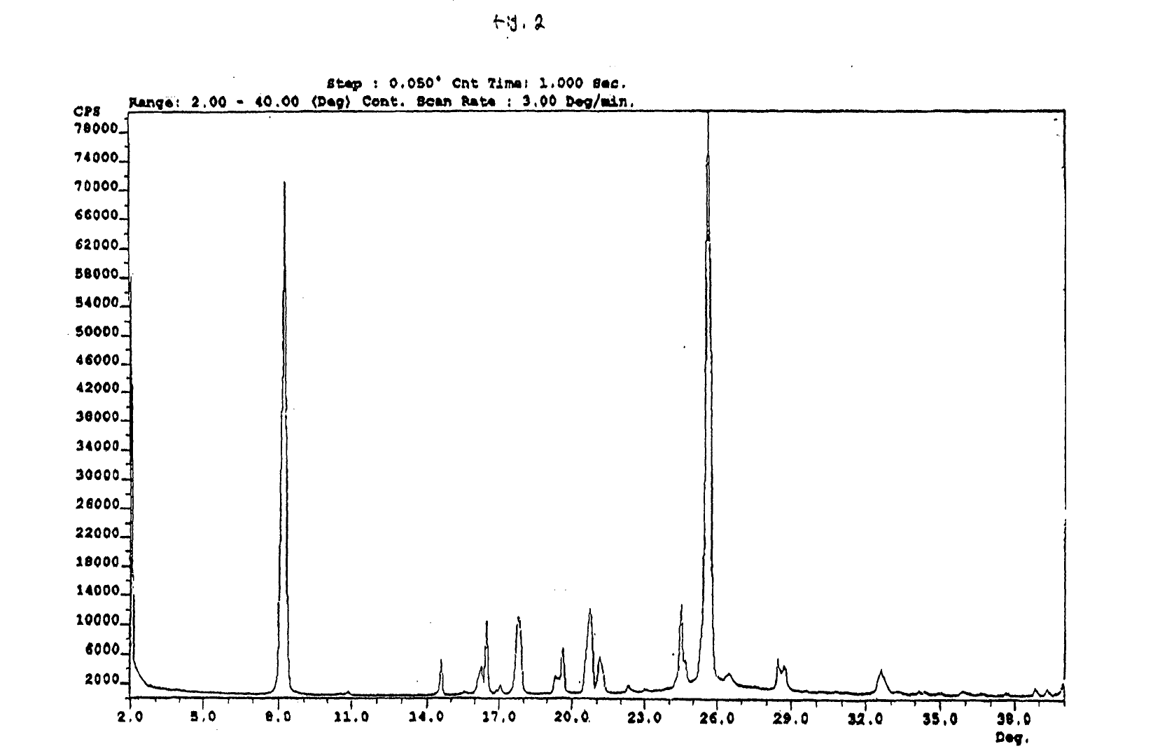

10. The crystalline solid famciclovir of claim 9, further characterized by an XRD pattern as substantially depicted in FIG. 2.

11. A crystalline solid famciclovir form III, characterized by an XRD pattern with peaks at 6.6 and 13.0±0.2 deg. 2θ.

12. The crystalline solid famciclovir of claim 11, further characterized by an XRD pattern with peaks at 15.9, 16.7, 18.4, 19.6, 24.5, 25.0 and 26.2±0.2 deg. 2θ.

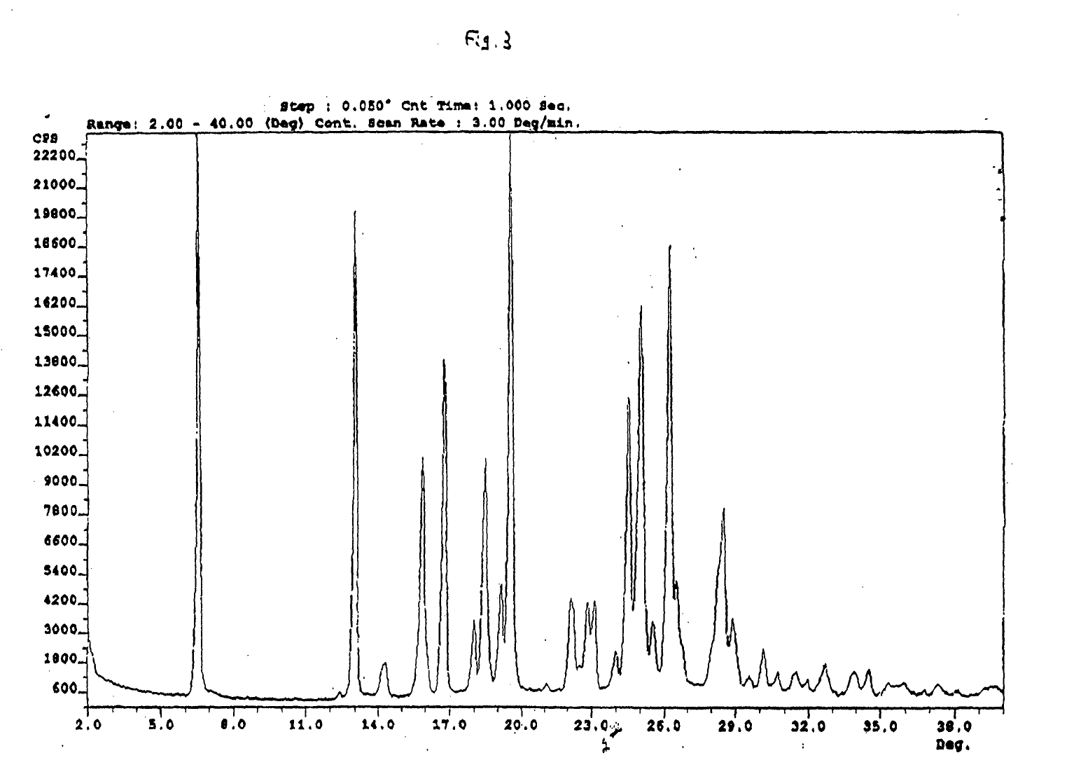

13. The crystalline solid famciclovir of claim 12, further characterized by an XRD pattern as substantially depicted in FIG. 3.

14. The crystalline solid famciclovir of claim 11, wherein the crystalline solid of famciclovir is a methanol solvate.

15. The crystalline solid famciclovir of claim 11, wherein the crystalline solid of famciclovir is an ethanol solvate.

16. Crystalline solid famciclovir methanol solvate.

17. Crystalline solid famciclovir ethanol solvate.

18. A process for preparing the crystalline solid famciclovir of claim 1, comprising the steps of:
a) triturating an anhydrous famciclovir form in an organic solvent selected from the group consisting of isopropyl alcohol, acetonitrile, and diethylether; and
b) isolating the crystalline solid famciclovir of claim 1.

19. A crystalline solid famciclovir form I prepared by triturating an anhydrous famciclovir form in an organic solvent selected from the group consisting of isopropyl alcohol, acetonitrile, and diethylether.

20. A process for preparing the crystalline solid famciclovir of claim 1, comprising the steps of:
a) heating crystalline solid famciclovir of claim 11 to about 40° C. to about 90° C.; and
b) isolating the crystalline solid famciclovir of claim 1.

21. The process of claim 20, wherein the heating of crystalline solid famciclovir of claim 11 is performed at a temperature of about 60° C. to about 70° C.

22. A process for preparing the crystalline solid famciclovir of claim 1, comprising the steps of:
a) heating famciclovir monohydrate to about 40° C. to about 80° C.; and
b) isolating the crystalline solid famciclovir of claim 1.

23. The process of claim 22, wherein the famciclovir monohydrate includes the crystalline solid famciclovir of claim 1.

24. The process of claim 22, wherein the heating of famciclovir monohydrate is performed at a temperature of about 60° C. to about 70° C.

25. A process for preparing the crystalline solid famciclovir of claim 1, comprising the steps of:
a) heating the crystalline solid famciclovir of claim 8 to about 40° C. to about 90° C.; and
b) isolating the crystalline solid famciclovir of claim 1.

26. The processes of any of claims 18, 20, 22 and 25, wherein the isolated crystalline solid famciclovir of claim 1 contains less than about 5% wt of other famciclovir crystalline forms.

27. The processes of any of claims 18, 20, 22, 25 and 26, wherein the isolated crystalline solid famciclovir of claim 1 contains less than about 5% wt of the form of claim 8.

28. The process of claim 26, wherein the isolated crystalline solid famciclovir of claim 1 contains less than about 1% wt of other famciclovir crystalline forms.

29. The process of claim 28, wherein the isolated crystalline solid famciclovir of claim 1 contains less than about 1% wt of the form of claim 8.

30. A process for preparing the crystalline solid famciclovir of claim 1, comprising the steps of:
a) providing a solution of famciclovir in an organic solvent selected from the group consisting of dichloromethane, chloroform, acetonitrile, ethylacetate, acetone, THF, diethyl ether/dichloromethane mixture, dichloromethane/toluene mixture, ethylacetate/toluene mixture, acetonitrile/toluene mixture, dimethylacetamide and isopropylalcohol;
b) cooling the solution; and
c) isolating the crystalline solid famciclovir of claim 1.

31. A process for preparing the crystalline solid famciclovir of claim 8, comprising the steps of:
a) providing a solution of famciclovir in an organic solvent selected from the group consisting of ethanol and n-buthanol,
b) cooling the solution whereby the crystalline solid famciclovir form II crystallizes, and
c) isolating the crystalline solid famciclovir of claim 8.

32. A process for preparing a mixture of crystalline solid famciclovir of claim 8 and crystalline solid famciclovir of claim 1 comprising the steps of:
a) providing a solution of famciclovir in an organic solvent selected from the group consisting of chloroform, ethylacetate, diethyl ether/dichloromethane mixture, tetrahydrofuran, acetonitrile/toluene mixture, dimethylacetamide and isopropanol,
b) cooling the solution, and
c) isolating the mixture of the crystalline solid famciclovir of claim 8 and the crystalline solid famciclovir of claim 1.

33. A process for preparing the crystalline solid famciclovir of claim 11, comprising the steps of:
a) triturating an anhydrous famciclovir in methanol; and
b) isolating the crystalline solid famciclovir of claim 11.

34. A process of preparing a mixture of the crystalline solid famciclovir of claim 11 and the crystalline solid famciclovir of claim 1, comprising the steps of:
a) triturating an anhydrous famciclovir in ethanol; and
b) isolating the mixture of the crystalline solid famciclovir of claim 11 and the crystalline solid famciclovir of claim 1.

35. A process of preparing a crystalline solid famciclovir monhydrate, comprising the steps of:
a) providing a solution of famciclovir in an organic solvent selected from the group consisting of acetonitrile, ethyl acetate, acetone, isopropyl alcohol, tetrahydrofuran, ethanol/water mixture, acetone/water mixture, DMF/water mixture, DMA/water mixture, acetonitrile/water mixture, methanol/water mixture, tetrahydrofuran/water mixture, and isopropyl alcohol/water mixture; and
b) cooling the solution; and
c) isolating the crystalline solid famciclovir monohydrate.

36. A process for preparing a mixture of the crystalline solid famciclovir of claim 11 and crystalline solid famciclovir monohydrate, comprising the steps of:
a) triturating anhydrous famciclovir in an organic solvent selected from the group consisting of isopropyl alcohol and ethanol; and
b) isolating the mixture of the crystalline solid famciclovir of claim 11 and crystalline solid famciclovir monohydrate.

37. A pharmaceutical composition comprising the crystalline solid famciclovir of claim 1 and a pharmaceutically-acceptable excipient, wherein the crystalline solid famciclovir of claim 1 contains less than about 5% wt of other famciclovir crystalline forms.

38. The pharmaceutical composition of claim 37, wherein the crystalline solid famciclovir of claim 1 contains less than about 1% wt of other famciclovir crystalline forms.

39. A pharmaceutical composition comprising the crystalline solid famciclovir of claim 8 and a pharmaceutically-acceptable excipient, wherein the crystalline solid famciclovir form II contains less than about 5% wt of other famciclovir crystalline forms.

40. The pharmaceutical composition of claim 39, wherein the crystalline solid famciclovir of claim 8 contains less than about 1% wt of other famciclovir crystalline forms.

41. A pharmaceutical composition comprising the crystalline solid famciclovir of claim 11 and a pharmaceutically-acceptable excipient, wherein the crystalline solid famciclovir form III contains less than about 5% wt of other famciclovir crystalline forms.

42. The pharmaceutical composition of claim 41, wherein the crystalline solid famciclovir of claim 11 contains less than about 1% wt of other famciclovir crystalline forms.

43. A method of treating a human in need of treatment with famciclovir comprising administering to the human the pharmaceutical composition of any of claims 37-42.
